SEQUENCE LISTING (6 sequences)

SEQ ID NO: 1 - protein, 357 aa, Homo sapiens
MEEGMNVLHDFGIQSTHYLQVNYQDSQDWFILVSVIADLRNAFYVLFPIWFHLQEAVGIKLLWVAVIGDWLNLVFKWILFGQRPYWWVLDTDYYSNTSVPLIKQFPVTCETGPGSPSGHAMGTAGVYYVMVTSTLSIFQGKIKPTYRFRCLNVILWLGFWAVQLNVCLSRIYLAAHFPHQVVAGVLSGIAVAETFSHIHSIYNASLKKYFLITFFLFSFAIGFYLLLKGLGVDLLWTLEKAQRWCEQPEWVHIDTTPFASLLKNLGTLFGLGLALNSSMYRESCKGKLSKWLPFRLSSIVASLVLLHVFDSLKPPSQVELVFYVLSFCKSAVVPLASVSVIPYCLAQVLGQPHKKSL

SEQ ID NO: 2 - protein, 357 aa, Homo sapiens
MEEGMNVLHDFGIQSTHYLQVNYQDSQDWFILVSVIADLRNAFYVLFPIWFHLQEAVGIKLLWVAVIGDWLNLVFKWILFGQRPYWWVLDTDYYSNTSVPLIKQFPVTCETGPGSPSGHAMGTAGVYYVMVTSTLSIFQGKIKPTYRFRCLNVILWLGFWAVQLNVCLSRIYLAAHFPHQVVAGVLSGIAVAETFSHIHSIYNASLKKYFLITFFLFSFAIGFYLLLKGLGVDLLWTLEKAQRWCEQPEWVHIDTTPFASLLKNLGTLFGLGLALNSSMYRESCKGKLSKWLPFRLGCIVASLVLLHLFDSLKPPSQVELIFYVLSFCKSAVVPLASVSLIPYCLAQVLGQPHKKSL

SEQ ID NO: 3 - protein, 357 aa, Homo sapiens
MEEGMNVLHDFGIQSTHYLQVNYQDSQDWFILVSVIADLRNAFYVLFPIWFHLQEAVGIKLLWVAVIGDWLNLVFKWILFGQRPYWWVLDTDYYSNTSVPLIKQFPVTCETGPGSPSGHAMGTAGVYYVMVTSTLSIFRGKKKPTYRFRCLNVILWLGFWAVQLNVCLSRIYLAAHFPHQVVAGVLSGIAVAETFSHIHSIYNASLKKYFLITFFLFSFAIGFYLLLKGLGVDLLWTLEKAQRWCERPEWVHIDTTPFASLLKNLGTLFGLGLALNSSMYRESCKGKLSKWLPFRLSCIVASLVLLHLFDSLKPPSQVELVFYVLSFCKSAVVPLASVSVIPYCLAQVLGQPHKKSL

SEQ ID NO: 4 - RNA, 1071 nt, Homo sapiens
auggaggaaggaaugaauguucuccaugacuuugggauccagucaacacauuaccuccaggugaauuaccaagacucccaggacugguucaucuugguguccgugaucgcagaccucaggaaugccuucuacguccucuuccccaucugguuccaucuucaggaagcugugggcauuaaacuccuuuggguagcugugauuggagacuggcucaaccucgucuuuaaguggauucucuuuggacagcguccauacugguggguuuuggauacugacuacuacagcaacacuuccgugccccugauaaagcaguucccuguaaccugugagacuggaccagggagccccucuggccaugccaugggcacagcagguguauacuacgugauggucacaucuacucuuuccaucuuucagggaaagauaaagccgaccuacagauuucggugcuugaaugucauuuugugguugggauucugggcugugcagcugaaugucugucugucacgaaucuaccuugcugcucauuuuccucaucaaguuguugcuggaguccugucaggcauugcuguugcagaaacuuucagccacauccacagcaucuauaaugccagccucaagaaauauuuucucauuaccuucuuccuguucagcuucgccaucggauuuuaucugcugcucaagggacuggguguagaccuccuguggacucuggagaaagcccagagguggugcgagcagccagaauggguccacauugacaccacacccuuugccagccuccucaagaaccugggcacgcucuuuggccuggggcuggcucucaacuccagcauguacagggagagcugcaaggggaaacucagcaaguggcucccauuccgccucagcucuauuguagccucccucguccuccugcacgucuuugacuccuugaaacccccaucccaagucgagcuggucuucuacgucuuguccuucugcaagagugcgguagugccccuggcauccgucagugucauccccuacugccucgcccagguccugggccagccgcacaagaagucguug

SEQ ID NO: 5 - RNA, 1070 nt, Homo sapiens
uggaggaaggaaugaauguucuccaugacuuugggauccagucaacacauuaccuccaggugaauuaccaagacucccaggacugguucaucuugguguccgugaucgcagaccucaggaaugccuucuacguccucuuccccaucugguuccaucuucaggaagcugugggcauuaaacuccuuuggguagcugugauuggagacuggcucaaccucgucuuuaaguggauucucuuuggacagcguccauacugguggguuuuggauacugacuacuacagcaacacuuccgugccccugauaaagcaguucccuguaaccugugagacuggaccagggagccccucuggccaugccaugggcacagcagguguauacuacgugauggucacaucuacucuuuccaucuuucagggaaagauaaagccgaccuacagauuucggugcuugaaugucauuuugugguugggauucugggcugugcagcugaaugucugucugucacgaaucuaccuugcugcucauuuuccucaucaaguuguugcuggaguccugucaggcauugcuguugcagaaacuuucagccacauccacagcaucuauaaugccagccucaagaaauauuuucucauuaccuucuuccuguucagcuucgccaucggauuuuaucugcugcucaagggacuggguguagaccuccuguggacucuggagaaagcccagagguggugcgagcagccagaauggguccacauugacaccacacccuuugccagccuccucaagaaccugggcacgcucuuuggccuggggcuggcucucaacuccagcauguacagggagagcugcaaggggaaacucagcaaguggcucccauuccgccucggcuguauuguagccucccucguccuccugcaccucuuugacuccuugaaacccccaucccaagucgagcugaucuucuacgucuuguccuucugcaagagugcgguagugccccuggcauccgucagucucauccccuacugccucgcccagguccugggccagccgcacaagaagucguug

SEQ ID NO: 6 - RNA, 1071 nt, Homo sapiens
auggaggaaggaaugaauguucuccaugacuuugggauccagucaacacauuaccuccaggugaauuaccaagacucccaggacugguucaucuugguguccgugaucgcagaccucaggaaugccuucuacguccucuuccccaucugguuccaucuucaggaagcugugggcauuaaacuccuuuggguagcugugauuggagacuggcucaaccucgucuuuaaguggauucucuuuggacagcguccauacugguggguuuuggauacugacuacuacagcaacacuuccgugccccugauaaagcaguucccuguaaccugugagacuggaccagggagccccucuggccaugccaugggcacagcagguguauacuacgugauggucacaucuacucuuuccaucuuuaggggaaagaaaaagccgaccuacagauuucggugcuugaaugucauuuugugguugggauucugggcugugcagcugaaugucugucugucacgaaucuaccuugcugcucauuuuccucaucaaguuguugcuggaguccugucaggcauugcuguugcagaaacuuucagccacauccacagcaucuauaaugccagccucaagaaauauuuucucauuaccuucuuccuguucagcuucgccaucggauuuuaucugcugcucaagggacuggguguagaccuccuguggacucuggagaaagcccagagguggugcgagaggccagaauggguccacauugacaccacacccuuugccagccuccucaagaaccugggcacgcucuuuggccuggggcuggcucucaacuccagcauguacagggagagcugcaaggggaaacucagcaaguggcucccauuccgccucagcuguauuguagccucccucguccuccugcaccucuuugacuccuugaaacccccaucccaagucgagcuggucuucuacgucuuguccuucugcaagagugcgguagugccccuggcauccgucagugucauccccuacugccucgcccagguccugggccagccgcacaagaagucguug

(19) United States
(12) Patent Application Publication
(10) Pub. No.: US 2020/0179494 A1
(43) Pub. Date: Jun. 11, 2020

(54) Modified mRNA Encoding a Glucose 6 Phosphatase and Uses Thereof

(71) Applicant: ModernaTX, Inc., Cambridge, MA (US)

(72) Inventors: Jeremiah Farelli, Cambridge, MA (US); Daniel Roseman, Cambridge, MA (US); Romesh R. Subramanian, Framingham, MA (US)

(21) Appl. No.: 16/631,585

(22) PCT Filed: Jul. 24, 2018
(86) PCT No.: PCT/US2018/043403

§ 371 Date: Jan. 16, 2020

Related U.S. Application Data
(60) Provisional application No. 62/536,000, filed on Jul. 24, 2017.

(51) Int. Cl. A61K 38/46

(52) U.S. Cl. CPC A61K 38/465; C12Y 301/03009

(57) ABSTRACT

Described herein are methods and compositions for treating a glucose 6 phosphatase deficiency based on mRNA therapy.

### CROSS-REFERENCE TO RELATED APPLICATIONS

This application claims priority to U.S. Provisional Appl. No. 62/536,000, filed Jul. 24, 2017, the contents of which is incorporated by reference herein in its entirety.

### BACKGROUND

Glycogen storage disease type 1a (GSD1a), also known as von Gierke's disease, is caused by homozygous or compound heterozygous mutation in the G6PC gene, which encodes glucose-6-phosphatase (G6Pase). GSD1a typically manifests during the first year of life with severe hypoglycemia and hepatomegaly caused by the accumulation of glycogen. Affected individuals exhibit growth retardation, delayed puberty, lactic acidemia, hyperlipidemia, hyperuricemia. Afflicted adults exhibit a high incidence of hepatic adenomas.

As there is currently no effective treatment for the underlying genetic defect that leads to G6Pase deficiency and related diseases and disorders, development of a targeted therapeutic agent is needed.

### SUMMARY

In one embodiment, the disclosure is directed to a method of treating a disease or disorder associated with a glucose-6-phosphatase deficiency in a subject comprising administering to the subject a therapeutically effective amount of a composition comprising a modified mRNA molecule encoding a glucose-6-phosphatase polypeptide or active fragment thereof. In a particular embodiment, the glucose-6-phosphatase polypeptide is encoded by G6PC. In a particular embodiment, the glucose-6-phosphatase polypeptide comprises an amino acid sequence that is at least about 80% identical, at least about 85% identical, at least about 90% identical or at least about 95% identical to a sequence selected from the group consisting of SEQ ID NOS:4-6. In a particular embodiment, the glucose-6-phosphatase polypeptide comprises an amino acid sequence selected from the group consisting of SEQ ID NOS:1-3. In a particular embodiment, the modified mRNA molecule comprises a sequence complementary to a nucleotide sequence that is at least about 80% identical, at least about 85% identical, at least about 90% identical or at least about 95% identical to a sequence selected from the group consisting of SEQ ID NOS:4-6. In a particular embodiment, the modified mRNA molecule comprises a sequence complementary to the nucleotide sequence selected from the group consisting of SEQ ID NOS:4-6. In a particular embodiment, the glucose-6-phosphatase deficiency is glycogen storage disease type 1a (von Gierke's disease). In a particular embodiment, the modified mRNA molecule comprises at least one modified nucleoside selected from the group consisting of: pseudouridine, 1-methyl pseudouridine, N1-methyl pseudouridine, 5-methylcytidine, 5-methyluridine, 2′-O-methyluridine, 2-thiouridine and N⁶-methyladenosine. In a particular embodiment, the modified mRNA molecule comprises a poly(A) tail, a Kozak sequence, a 3′ untranslated region, a 5′ untranslated region or any combination thereof.

In one embodiment, the disclosure is directed to a pharmaceutical composition comprising a therapeutically effective amount of a modified mRNA molecule encoding a glucose-6-phosphatase polypeptide, active variant of a glucose-6-phosphatase polypeptide, or active fragment thereof formulated in a lipid nanoparticle carrier.

In one embodiment, the disclosure is directed to a method of reducing glycogen levels in a subject comprising administering a therapeutically effective amount of a modified mRNA capable of expressing a glucose-6-phosphatase polypeptide or biologically active fragment thereof. In a particular embodiment, the glucose-6-phosphatase polypeptide is encoded by G6PC. In a particular embodiment, the glucose-6-phosphatase polypeptide comprises an amino acid sequence that is at least about 80% identical, at least about 85% identical, at least about 90% identical or at least about 95% identical to a sequence selected from the group consisting of SEQ ID NOS:1-3.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a drawing showing a disease overview caused by G6Pase deficiency. A G6Pase deficiency leads to an inability to produce blood glucose (e.g., lack of glycogen mobilization and gluconeogenesis), complete metabolic derangement (e.g., hypoglycemia, accumulation of glycogen in the liver, elevated levels of lactate, uric acid, triglycerides and cholesterol) and long-term complications (e.g., hepatocellular adenomas, renal dysfunction, anemia, osteoporosis and gout).

FIG. 2 shows western blot analysis of G6Pase expression over time in human primary hepatocytes transfected with WT or protein engineered modRNAs. Cells transfected with huG6PC-wt (WT), huG6PC-V1 (variant 1), and huG6PC-V (variant 2) modRNAs were lysed at the indicated days post-transfection and analyzed via western blot using a rabbit antihuman G6Pase PAb.

FIG. 3 is a graph showing G6Pase enzyme activity in mouse livers 96 hours after mRNA therapy. G6Pase activity was measured in microsomal preparations from frozen livers of GFP-treated WT (left circles), GFP-treated KO (second from left circles), G6PC-WT treated KO (third from left circles), G6PC-V1 treated KO (fourth from left circles), and G6PC-V2 treated KO mice (fifth from left circles). Data were obtained from mice fasted for 24 hr and individual treatment groups (n=5 or 6) are shown with bars representing the mean±95% CI. The groups were compared by one-way ANOVA followed by Dunnetts comparison test. Values significantly different from GFP-treated KO mice (*P<0.05, **P<0.005, and ***P<0.0005) are indicated.

FIG. 4 shows a series of plots showing hepatic biomarkers are significantly lowered at four days post-dose of hG6PC. (Panel A) Liver weight as % of total body weight; (Panel B) hepatic G6P content; (Panel C) hepatic glycogen content; (Panel D) hepatic triglyceride content in GFP-treated WT (left circles), GFP-treated KO (second from left circles), G6PC-WT treated KO (third from left circles), G6PC-V1 treated KO (fourth from left circles), and G6PC-V2 treated KO mice (fifth from left circles). Data were obtained from mice fasted for 24 hr and individual treatment groups (n=5 or 6) are shown with bars representing the mean±95% CI. The groups were compared by one-way ANOVA followed by Dunnetts comparison test. Values significantly different from GFP-treated KO mice (*P<0.05, **P<0.005, and ***P<0.0005) are indicated.

FIGS. 5A-5C are graphs showing the effect of mRNA therapy after 72 hr on fasting glycaemia. FIG. 5A (top) is a plot of fasting blood glucose levels out to 9 hours in GFP-treated WT (second from top circles at nine hours), GFP-treated KO (bottom circles at nine hours), G6PC-WT treated KO (fourth from top circles at nine hours), G6PC-V1 treated KO (third from top circles at nine hours) and G6PC-V2 treated KO mice (top circles at nine hours). Areas under the curves (AUC) were calculated from the plots and individual treatment groups (n=5 or 6) are shown with bars representing the mean±95% CI in the lower panel. FIG. 5B shows results after 6 days (GFP treated WT (top circles at nine hours), GFP treated KO (bottom circles at nine hours), G6PC WT treated KO (fourth from top circles at nine hours), G6PC V1 treated KO (third from top circles at nine hours) and G6PC V2 treated KO mice (second from top circles at nine hours)). FIG. 5C shows results after 11 days (GFP treated WT (top circles at nine hours), GFP treated KO (bottom circles at nine hours), G6PC WT treated KO (fourth from top circles at nine hours), G6PC V1 treated KO (third from top circles at nine hours) and G6PC V2 treated KO mice (second from top circles at nine hours)). The groups were compared by one-way ANOVA followed by Dunnetts comparison test. Values significantly different from GFP-treated KO mice (*P<0.05, **P<0.005, and ***P<0.0005) are indicated.

FIGS. 6A-6B are plots showing the effect of mRNA therapy after 7 and 12 days on liver weight. Liver weight as % of total body weight after (FIG. 6A) 7 days or (FIG. 6B) 12 days post-dose. GFP-treated WT (left circles), GFP-treated KO (second from left circles), G6PC-WT treated KO (third from left circles), G6PC-V1 treated KO (fourth from left circles), and G6PC-V2 treated KO mice (fifth from left circles). Data were obtained from mice fasted for 24 hr and individual treatment groups (n=5 or 6) are shown with bars representing the mean±95% CI. The groups were compared by one-way ANOVA followed by Dunnetts comparison test. Values significantly different from GFP-treated KO mice (*P<0.05, **P<0.005, and ***P<0.0005) are indicated.

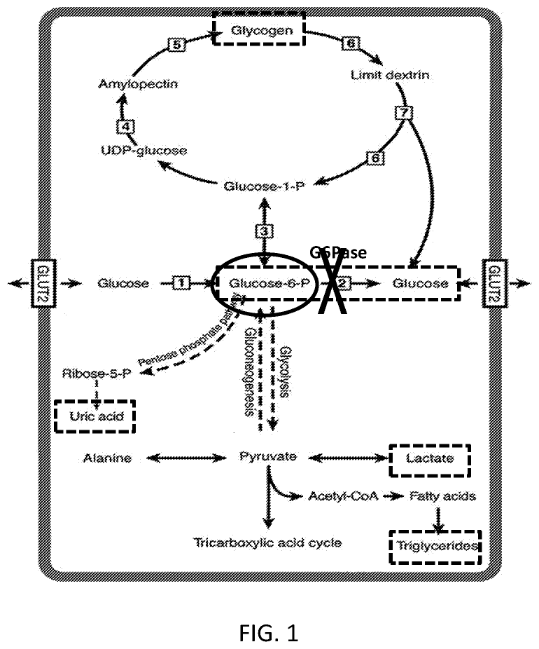

### DETAILED DESCRIPTION

Compositions and methods are described herein to treat or ameliorate a disease, disorder or condition associated with a glucose-6-phosphatase (G6Pase) deficiency, accumulation of glycogen, and elevated or deficient levels of molecular markers associated with a G6Pase deficiency, comprising administering to a subject a composition comprising a nucleic acid, e.g., a messenger RNA molecule, e.g., modified or unmodified, encoding a G6Pase polypeptide.

As used herein, the term “messenger RNA” (mRNA) refers to a polynucleotide that encodes a polypeptide of interest and is capable of being translated to produce the encoded polypeptide of interest in vitro, in vivo, in situ or ex vivo. As used herein, “disease” refers to any deviation from the normal health of a subject and includes a state when disease symptoms are present, as well as conditions in which a deviation has occurred, but symptoms are not yet manifested (e.g., a predecease condition). As used herein, “treatment” or “treat” refer to both therapeutic treatment and prophylactic or preventative measures. Those in need of treatment include those having a disorder as well as those at risk for a disease or disorder, or those in whom the disorder is to be prevented.

Described herein are nucleic acid molecules, including modified nucleic acid molecules, and methods of using the same. The nucleic acid molecules, including RNAs such as mRNAs, can comprise, for example, one or more modifications that improve properties of the molecule. Such improvements include, but are not limited to, increased stability and/or clearance in tissues, improved receptor uptake and/or kinetics, improved cellular access by the compositions, improved engagement with translational machinery, improved mRNA half-life, increased translation efficiency, improved immune evasion, improved protein production capacity, improved secretion efficiency, improved accessibility to circulation, improved protein half-life and/or modulation of a cell's status, improved function and/or improved activity.

The present disclosure provides compositions of nucleic acids capable of expressing or regulating protein expression of G6Pase or a biologically active fragment thereof in a target cell. Methods and processes of preparing and delivering such nucleic acid to a target cell are also provided. Kits and devices for the design, preparation, manufacture and formulation of such nucleic acids are also included in the instant disclosure.

The compositions provided herein are useful for treating a disease or disorder associated with a deficiency of G6Pase activity, such as, for example, glycogen storage disease type 1a (GSD1a). There are eleven distinct diseases that are commonly considered to be glycogen storage diseases, and type I (sometimes called con Gierke's disease) is characterized by a loss of function of G6Pase. The two subtypes (GSDIa and GSDIb) are clinically indistinguishable. GSDIa is caused by the deficiency of G6Pase catalytic activity; while GSDIb is caused by a defect in glucose-6-phosphate exchanger SLC37A4.

Nucleic acids useful for the present disclosure include, for example, polynucleotides, which further include, for example, ribonucleic acids (RNAs), deoxyribonucleic acids (DNAs), threose nucleic acids (TNAs; Yu, H. et al., Nat. Chem., 4:183-7, 2012), glycol nucleic acids (GNAs, for reviews see Ueda, N. et aL, J. Heterocyclic Chem., 8:827-9, 1971; Zhang, L. et al., J. Am. Chem. Soc., 127:4174-5, 2005), peptide nucleic acids (PNAs, see Nielsen, P. et al., Science, 254:1497-500, 1991), locked nucleic acids (LNAs; Koshkin, A. et al., Tetrahedron, 54:3607-30, 1998), and other polynucleotides known in the art.

The nucleic acid molecule can be a messenger RNA (mRNA), e.g., a modified mRNA (“modRNA), which encodes, for example, a G6Pase (e.g., encoded by the G6PC3 gene) or a biologically active fragment thereof. The mRNA can be delivered into a target cell, for example, to express a G6Pase or a biologically active fragment thereof. The mRNA can be translated in vivo, in situ or ex vivo.

The mRNA can be administered to an animal, e.g., a mammal (such as a human), to express a G6Pase polypeptide or a biologically active fragment thereof. The mRNA provided is capable, for example, of treating or alleviating a symptom, a disease or a disorder associated with a deficiency of G6Pase activity, such as, for example, a GSD, e.g., GSD1a.

### RNA Structure

Modified mRNA molecules are described herein that provide for a therapeutic tool for use in enzyme replacement therapy (ERT), e.g., for treating GSD1a or a disease or condition associated with G6Pase deficiency. The terms “modified” or “modification” as used herein refer to an alteration of a canonical nucleic acid residue that can be, for example, incorporated into a polynucleotide, e.g., an mRNA molecule, which can then be used for a therapeutic treatment. Modifications to an mRNA molecule can include, for example, physical or chemical modifications to a base, such as, for example, the depletion of a base or a chemical modification of a base, or sequence modifications to a nucleic acid sequence relative to a reference nucleic acid sequence.

Described herein are compositions for modulating the expression of a G6Pase or a biologically active fragment thereof in vitro or in vivo, e.g., in a target cell. The mRNA molecule can, for example, replace, increase or promote expression of such a G6Pase or biologically active fragment thereof. In some embodiments, the composition comprises an artificially synthesized or isolated nature RNA molecule with or without a transfer vehicle. An RNA molecule can comprise, for example, a sequential series of sequence elements, wherein, for example, a coding sequence comprises a nucleic acid sequence encoding a G6Pase or a biologically active fragment thereof. The coding sequence can comprise, with or without a bridging linker (such as a peptide linker comprising at least one amino acid residue), one or more 5′ signal sequence(s). A sequence upstream of the coding sequence can comprise an optional flanking region comprising one or more complete or incomplete 5′ untranslated region (UTR) sequences. A sequence upstream of the optional flanking sequence can comprise an optional 5′ terminal cap. A sequence downstream of the coding sequence can comprise an optional flanking region comprising one or more complete or incomplete 3′ UTR sequences. A sequence downstream of the optional 3′ flanking sequence can comprise an optional flanking region comprising a 3′ tailing sequence. Bridging the 5′ terminus of the coding sequence and the upstream flanking sequence is an optional first operational region. This first operational region traditionally comprises a start codon. The operational region can also comprise, for example, a translation initiation sequence or signal sequence. Bridging the 3′ end of the coding sequence and the 3′ downstream flanking sequence is an optional second operational region. This second operational region can comprise, for example, a stop codon. The second 3′ operational sequence can also comprise a translation termination sequence or signal sequence. Multiple, serial stop codons can also be used. The 3′ terminus of the RNA can comprise a 3′ tail sequence, e.g., a poly-A tail.

UTRs are transcribed but not translated. The 5′ UTR starts at the transcription start site and continues to the start codon but does not include the start codon; whereas, the 3′ UTR starts immediately following the stop codon and continues until the transcriptional termination signal. Natural 5′ UTRs help translation initiation, and they comprise features such as, for example, Kozak sequences, which facilitate translation initiation by the ribosome for many genes. Kozak sequences have the consensus CCRCCAUGG, where R is a purine (adenine or guanine) three bases upstream of the start codon (AUG), which is followed by another G.

3′ UTRs are rich in adenosines and uridines. These AU-rich signatures are particularly prevalent in genes with high rates of turnover. Based on their sequence features and functional properties, the AU-rich elements (AREs) can be separated into three classes—Class I AREs (such as those in c-Myc and MyoD) contain several dispersed copies of an AUUUA motif within U-rich regions; Class II AREs possess two or more overlapping UUAUUUA(U/A)(U/A) nonamers (molecules containing this type of ARE include GM-CSF and TNFα); Class III ARES are less well defined (these U-rich regions do not contain an AUUUA motif; c-Jun and myogenin are two examples of this class). Most proteins binding to the AREs destabilize the messenger, whereas members of the ELAV family, most notably HuR, increase the stability of mRNA. Engineering HuR specific binding site(s) into the 3′ UTR of the mRNA leads to HuR binding and thus, stabilization of the mRNA.

Introduction, removal or modification of 3′ UTR AREs can be used to modulate the stability of mRNA. When engineering specific mRNA, one or more copies of an ARE can be introduced to make such mRNA less stable and thereby curtail translation and decrease production of the resultant protein. Likewise, AREs can be identified and removed or mutated to increase the intracellular stability and thus increase translation and production of the resultant protein.

The 5′ cap structure of an mRNA is involved in nuclear export and mRNA stability in the cell. The cap binds to Cap Binding Protein (CBP), which is responsible for in vivo mRNA stability and translation competency through the interaction of CBP with poly-A binding protein to form the mature cyclic mRNA species. The cap further assists the removal of 5′ proximal introns during mRNA splicing. The mRNA molecules of the instant disclosure may be 5′ end capped to generate a 5′-ppp-5′-triphosphate linkage. The linkage site is between a terminal guanosine cap residue and the 5′-terminal transcribed sense nucleotide of the mRNA molecule. This 5′-guanylate cap may then be methylated to generate an N⁷-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

mRNA can be capped post-transcriptionally, for example, using enzymes to generate more authentic 5′ cap structures. As used herein, the phrase “more authentic” refers to a feature that closely mirrors or mimics, either structurally or functionally, a naturally occurring feature. That is, a “more authentic” feature is better representative of physiological cellular function and/or structure as compared to synthetic features or analogs. Non-limiting examples of more authentic 5′ cap structures are those that, among other things, have enhanced binding of CBPs, increased half-life, reduced susceptibility to 5′ endonucleases and/or reduced 5′ decapping, as compared to synthetic 5′ cap structures. Recombinant Vaccinia virus capping enzyme and recombinant 2′-O-methyltransferase, for example, can create a canonical 5′-5′-triphosphate linkage between the 5′ terminal nucleotide of an mRNA and a guanine cap nucleotide wherein the cap guanine contains an N7 methylation and the 5′ terminal nucleotide of the mRNA contains a 2′-O-methyl. Such a structure is termed the “Cap1” structure. This cap results in a higher translational competency and cellular stability and a reduced activation of cellular pro-inflammatory cytokines, as compared, for example, to other 5′-cap analog structures. Because the mRNA of the instant disclosure may be capped post-transcriptionally, and because this process is more efficient, nearly 100% of the mRNA may be capped. This is in contrast to the ˜80% capping rate when a cap analog is linked to an mRNA in the course of an in vitro transcription reaction.

Cap analogs can be used to modify the 5′ end of an mRNA molecule. Cap analogs, synthetic cap analogs, chemical caps, chemical cap analogs, or structural or functional cap analogs, differ from natural 5′ caps in their chemical structure, while still retaining cap function. Cap analogs can be chemically or enzymatically synthesized and/or linked to the mRNA, e.g., modRNA, described herein. The Anti-Reverse Cap Analog (ARCA), for example, contains two guanines linked by a 5′-5′-triphosphate group, wherein one guanine contains an N⁷ methyl group as well as a 3′-O-methyl group. Another exemplary cap is mCAP, which is similar to ARCA but has a 2′-O-methyl group on guanosine. Cap structures include, but are not limited to, 5′ triphosphate cap (5′-ppp), Guanosine-triphosphate Cap (5′-Gppp), 5′ N⁷-methylguanosine-triphosphate Cap (5′ N⁷-MeGppp, 7mGppp), 5′ Adenylated cap (rApp), 7mG(5′)ppp(5′)N, pN2p (cap 0), 7mG(5′)ppp(5′)NlmpNp (cap 1), and 7mG(5′)-ppp(5′)NlmpN2mp (cap 2) (Konarska, M. et al., Cell, 38:731-6, 1984; the entire contents of which are incorporated by reference). A 5′ terminal cap can further comprise a guanine analog. Useful guanine analogs include, but are not limited to, inosine, N′-methyl-guanosine, 2′-fluoro-guanosine, 7-deaza-guanosine, 8-oxo-guanosine, 2-amino-guanosine, LNA-guanosine and 2-azido-guanosine.

### RNA Sequence

Described herein are modRNA sequences encoding a G6Pase or a biologically active fragment thereof, which is useful for, among other things, treating a disease or disorder associated with a deficiency of G6Pase activity, such as, for example, GSDIa. As used herein, a “biologically active fragment” refers to a portion of a molecule, e.g., a gene, coding sequence, mRNA, polypeptide or protein, which has a desired length or biological function. A biologically active fragment of a protein, for example, can be a fragment of the full-length protein that retains one or more biological activities of the protein. A biologically active fragment of an mRNA, for example, can be a fragment that, when translated, expresses a biologically active protein fragment. A biologically active mRNA fragment, furthermore, can comprise shortened versions of non-coding sequences, e.g., regulatory sequences, UTRs, etc. In general, a fragment of an enzyme or signaling molecule can be, for example, that portion(s) of the molecule that retains its signaling or enzymatic activity. A fragment of a gene or coding sequence, for example, can be that portion of the gene or coding sequence that produces an expression product fragment. As used herein, “gene” is a term used to describe a genetic element that gives rise to expression products (e.g., pre-mRNA, mRNA, polypeptides etc.). A fragment does not necessarily have to be defined functionally, as it can also refer to a portion of a molecule that is not the whole molecule, but has some desired characteristic or length (e.g., restriction fragments, amplification fragments, etc.).

Additional sequence modification, for example to the 3′ UTR, include the insertion of, for example, viral sequences such as the translation enhancer sequence of the barley yellow dwarf virus (BYDV-PAV), the Jaagsiekte sheep retrovirus (JSRV) and/or the Enzootic nasal tumor virus (PCT Pub. No. WO2012129648; herein incorporated by reference in its entirety).

modRNA described herein can comprise an internal ribosome entry site (IRES). IRESs play an important role in initiating protein synthesis in absence of the 5′ cap structure. An IRES can act as the sole ribosome binding site, or serve as one of multiple ribosome binding sites of an mRNA. An mRNA containing more than one functional ribosome binding site can encode several peptides or polypeptides that are translated independently by the ribosomes (“multicistronic nucleic acid molecules”). A modRNA can thus encode, for example, multiple portions or fragments of a G6Pase or a biologically active fragment thereof. Examples of IRES sequences that can be used include IRESs derived from, for example, picornaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) and cricket paralysis viruses (CrPV).

During RNA processing, a long chain of adenine nucleotides (poly-A tail) can be added to the mRNA molecule. The process, called polyadenylation, adds a poly-A tail that can be between, for example, about 100 and 250 residues long. In some embodiments, unique poly-A tail lengths provide certain advantages to the mRNA of the instant disclosure. Generally, the length of a poly-A tail is greater than 30 nucleotides in length (e.g., at least or greater than about 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the mRNA comprises a poly-A tail of a length from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000). In some embodiments, the poly-A tail is designed relative to the length of the overall mRNA. This design may be based on the length of the coding region, the length of a particular feature or region (such as the first or flanking regions), or based on the length of the ultimate product expressed from the mRNA. The poly-A tail can be, for example, 10, 20, 30, 40, 50, 60, 70, 80, 90 or 100% greater in length than the rest of the mRNA sequence. The poly-A tail can also be designed as a fraction of such mRNA.

mRNA can be linked together to the PABP (Poly-A binding protein) through the 3′ end using modified nucleotides at the 3′ terminus of the poly-A tail. In one embodiment, mRNA can include a poly-A tail-G-quartet. The G-quartet is a cyclic hydrogen bonded array of four guanine nucleotides that can be formed by G-rich sequences in both DNA and RNA. In this embodiment, the G-quartet is incorporated at the end of the poly-A tail.

Other RNA sequence modification elements and methods include a combination of nucleotide modifications abrogating mRNA interaction with Toll-like receptor 3 (TLR3), TLR7, TLR8 and retinoid-inducible gene 1 (RIG-1), resulting in low immunogenicity and higher stability in mice (Kormann, M. et al., Nat. Biotechnol., 29:154-7, 2011; the content of which is incorporated by reference herein in its entirety).

### Glucose-6-Phosphatase

Glucose-6-phosphatase is a complex of multiple component proteins, including transporters for G6P, glucose and phosphate. The main phosphatase function is performed by the glucose-6-phosphatase catalytic subunit. In humans, there are three isozymes of the catalytic subunit: glucose-6-phosphatase-α, encoded by G6PC; IGRP, encoded by G6PC2; and glucose-6-phosphatase-β, encoded by G6PC3. G6Pase-α and G6Pase-β are both functional phosphohydrolases, and have similar active site structure, topology, mechanism of action and kinetic properties with respect to G6P hydrolysis.

Exemplary G6Pase sequences are shown below. Modifications to the sequences can occur as described herein, for example, by using modified or non-naturally occurring uracil residues throughout the mRNA sequence.

### G6Pase, Wild-Type Amino Acid Sequence:

(SEQ ID NO: 1)
MEEGMNVLHDFGIQSTHYLQVNYQDSQDWFILVSVIADLRNAFYVLFPI
WFHLQEAVGIKLLWVAVIGDWLNLVFKWILFGQRPYWWVLDTDYYSNTS
VPLIKQFPVTCETGPGSPSGHAMGTAGVYYVMVTSTLSIFQGKIKPTYR
FRCLNVILWLGFWAVQLNVCLSRIYLAAHFPHQVVAGVLSGIAVAETFS
HIHSIYNASLKKYFLITFFLFSFAIGFYLLLKGLGVDLLWTLEKAQRWC
EQPEWVHIDTTPFASLLKNLGTLFGLGLALNSSMYRESCKGKLSKWLPF
RLSSIVASLVLLHVFDSLKPPSQVELVFYVLSFCKSAVVPLASVSVIPY
CLAQVLGQPHKKSL

### G6Pase, Variant 1 Amino Acid Sequence:

(SEQ ID NO: 2)
MEEGMNVLHDFGIQSTHYLQVNYQDSQDWFILVSVIADLRNAFYVLFPI
WFHLQEAVGIKLLWVAVIGDWLNLVFKWILFGQRPYWWVLDTDYYSNTS
VPLIKQFPVTCETGPGSPSGHAMGTAGVYYVMVTSTLSIFQGKIKPTYR
FRCLNVILWLGFWAVQLNVCLSRIYLAAHFPHQVVAGVLSGIAVAETFS
HIHSIYNASLKKYFLITFFLFSFAIGFYLLLKGLGVDLLWTLEKAQRWC
EQPEWVHIDTTPFASLLKNLGTLFGLGLALNSSMYRESCKGKLSKWLPF
RLGCIVASLVLLHLFDSLKPPSQVELIFYVLSFCKSAVVPLASVSLIPY
CLAQVLGQPHKKSL

### G6Pase, Variant 2, Amino Acid Sequence

(SEQ ID NO: 3)
MEEGMNVLHDFGIQSTHYLQVNYQDSQDWFILVSVIADLRNAFYVLFPI
WFHLQEAVGIKLLWVAVIGDWLNLVFKWILFGQRPYWWVLDTDYYSNTS
VPLIKQFPVTCETGPGSPSGHAMGTAGVYYVMVTSTLSIFRGKKKPTYR
FRCLNVILWLGFWAVQLNVCLSRIYLAAHFPHQVVAGVLSGIAVAETFS
HIHSIYNASLKKYFLITFFLFSFAIGFYLLLKGLGVDLLWTLEKAQRWC
ERPEWVHIDTTPFASLLKNLGTLFGLGLALNSSMYRESCKGKLSKWLPF
RLSCIVASLVLLHLFDSLKPPSQVELVFYVLSFCKSAVVPLASVSVIPY
CLAQVLGQPHKKSL

### G6Pase, Wild-Type Coding Sequence:

SEQ ID NO: 4)
AUGGAGGAAGGAAUGAAUGUUCUCCAUGACUUUGGGAUCCAGUCAACAC
AUUACCUCCAGGUGAAUUACCAAGACUCCCAGGACUGGUUCAUCUUGGU
GUCCGUGAUCGCAGACCUCAGGAAUGCCUUCUACGUCCUCUUCCCCAUC
UGGUUCCAUCUUCAGGAAGCUGUGGGCAUUAAACUCCUUUGGGUAGCUG
UGAUUGGAGACUGGCUCAACCUCGUCUUUAAGUGGAUUCUCUUUGGACA
GCGUCCAUACUGGUGGGUUUUGGAUACUGACUACUACAGCAACACUUCC
GUGCCCCUGAUAAAGCAGUUCCCUGUAACCUGUGAGACUGGACCAGGGA
GCCCCUCUGGCCAUGCCAUGGGCACAGCAGGUGUAUACUACGUGAUGGU
CACAUCUACUCUUUCCAUCUUUCAGGGAAAGAUAAAGCCGACCUACAGA
UUUCGGUGCUUGAAUGUCAUUUUGUGGUUGGGAUUCUGGGCUGUGCAGC
UGAAUGUCUGUCUGUCACGAAUCUACCUUGCUGCUCAUUUUCCUCAUCA
AGUUGUUGCUGGAGUCCUGUCAGGCAUUGCUGUUGCAGAAACUUUCAGC
CACAUCCACAGCAUCUAUAAUGCCAGCCUCAAGAAAUAUUUUCUCAUUA
CCUUCUUCCUGUUCAGCUUCGCCAUCGGAUUUUAUCUGCUGCUCAAGGG
ACUGGGUGUAGACCUCCUGUGGACUCUGGAGAAAGCCCAGAGGUGGUGC
GAGCAGCCAGAAUGGGUCCACAUUGACACCACACCCUUUGCCAGCCUCC
UCAAGAACCUGGGCACGCUCUUUGGCCUGGGGCUGGCUCUCAACUCCAG
CAUGUACAGGGAGAGCUGCAAGGGGAAACUCAGCAAGUGGCUCCCAUUC
CGCCUCAGCUCUAUUGUAGCCUCCCUCGUCCUCCUGCACGUCUUUGACU
CCUUGAAACCCCCAUCCCAAGUCGAGCUGGUCUUCUACGUCUUGUCCUU
CUGCAAGAGUGCGGUAGUGCCCCUGGCAUCCGUCAGUGUCAUCCCCUAC
UGCCUCGCCCAGGUCCUGGGCCAGCCGCACAAGAAGUCGUUG

### G6Pase, Variant 1, Coding Sequence:

(SEQ ID NO: 5)
UGGAGGAAGGAAUGAAUGUUCUCCAUGACUUUGGGAUCCAGUCAACACA
UUACCUCCAGGUGAAUUACCAAGACUCCCAGGACUGGUUCAUCUUGGUG
UCCGUGAUCGCAGACCUCAGGAAUGCCUUCUACGUCCUCUUCCCCAUCU
GGUUCCAUCUUCAGGAAGCUGUGGGCAUUAAACUCCUUUGGGUAGCUGU
GAUUGGAGACUGGCUCAACCUCGUCUUUAAGUGGAUUCUCUUUGGACAG
CGUCCAUACUGGUGGGUUUUGGAUACUGACUACUACAGCAACACUUCCG
UGCCCCUGAUAAAGCAGUUCCCUGUAACCUGUGAGACUGGACCAGGGAG
CCCCUCUGGCCAUGCCAUGGGCACAGCAGGUGUAUACUACGUGAUGGUC
ACAUCUACUCUUUCCAUCUUUCAGGGAAAGAUAAAGCCGACCUACAGAU
UUCGGUGCUUGAAUGUCAUUUUGUGGUUGGGAUUCUGGGCUGUGCAGCU
GAAUGUCUGUCUGUCACGAAUCUACCUUGCUGCUCAUUUUCCUCAUCAA
GUUGUUGCUGGAGUCCUGUCAGGCAUUGCUGUUGCAGAAACUUUCAGCC
ACAUCCACAGCAUCUAUAAUGCCAGCCUCAAGAAAUAUUUUCUCAUUAC
CUUCUUCCUGUUCAGCUUCGCCAUCGGAUUUUAUCUGCUGCUCAAGGGA
CUGGGUGUAGACCUCCUGUGGACUCUGGAGAAAGCCCAGAGGUGGUGCG
AGCAGCCAGAAUGGGUCCACAUUGACACCACACCCUUUGCCAGCCUCCU
CAAGAACCUGGGCACGCUCUUUGGCCUGGGGCUGGCUCUCAACUCCAGC
AUGUACAGGGAGAGCUGCAAGGGGAAACUCAGCAAGUGGCUCCCAUUCC
GCCUCGGCUGUAUUGUAGCCUCCCUCGUCCUCCUGCACCUCUUUGACUC
CUUGAAACCCCCAUCCCAAGUCGAGCUGAUCUUCUACGUCUUGUCCUUC
UGCAAGAGUGCGGUAGUGCCCCUGGCAUCCGUCAGUCUCAUCCCCUACU
GCCUCGCCCAGGUCCUGGGCCAGCCGCACAAGAAGUCGUUG

### G6Pase, Variant 2, Coding Sequence:

(SEQ ID NO: 6)
AUGGAGGAAGGAAUGAAUGUUCUCCAUGACUUUGGGAUCCAGUCAACAC
AUUACCUCCAGGUGAAUUACCAAGACUCCCAGGACUGGUUCAUCUUGGU
GUCCGUGAUCGCAGACCUCAGGAAUGCCUUCUACGUCCUCUUCCCCAUC
UGGUUCCAUCUUCAGGAAGCUGUGGGCAUUAAACUCCUUUGGGUAGCUG
UGAUUGGAGACUGGCUCAACCUCGUCUUUAAGUGGAUUCUCUUUGGACA
GCGUCCAUACUGGUGGGUUUUGGAUACUGACUACUACAGCAACACUUCC
GUGCCCCUGAUAAAGCAGUUCCCUGUAACCUGUGAGACUGGACCAGGGA
GCCCCUCUGGCCAUGCCAUGGGCACAGCAGGUGUAUACUACGUGAUGGU
CACAUCUACUCUUUCCAUCUUUAGGGGAAAGAAAAAGCCGACCUACAGA
UUUCGGUGCUUGAAUGUCAUUUUGUGGUUGGGAUUCUGGGCUGUGCAGC
UGAAUGUCUGUCUGUCACGAAUCUACCUUGCUGCUCAUUUUCCUCAUCA
AGUUGUUGCUGGAGUCCUGUCAGGCAUUGCUGUUGCAGAAACUUUCAGC
CACAUCCACAGCAUCUAUAAUGCCAGCCUCAAGAAAUAUUUUCUCAUUA
CCUUCUUCCUGUUCAGCUUCGCCAUCGGAUUUUAUCUGCUGCUCAAGGG
ACUGGGUGUAGACCUCCUGUGGACUCUGGAGAAAGCCCAGAGGUGGUGC
GAGAGGCCAGAAUGGGUCCACAUUGACACCACACCCUUUGCCAGCCUCC
UCAAGAACCUGGGCACGCUCUUUGGCCUGGGGCUGGCUCUCAACUCCAG
CAUGUACAGGGAGAGCUGCAAGGGGAAACUCAGCAAGUGGCUCCCAUUC
CGCCUCAGCUGUAUUGUAGCCUCCCUCGUCCUCCUGCACCUCUUUGACU
CCUUGAAACCCCCAUCCCAAGUCGAGCUGGUCUUCUACGUCUUGUCCUU
CUGCAAGAGUGCGGUAGUGCCCCUGGCAUCCGUCAGUGUCAUCCCCUAC
UGCCUCGCCCAGGUCCUGGGCCAGCCGCACAAGAAGUCGUUG

In some embodiments, the G6Pase or biologically active fragment thereof, encoded by the mRNA described herein, comprises a protein sequence with at least 70%, 75%, 80%, 85%, 90%, 91%, 92%, 93%, 94%, 95%, 96%, 97%, 98%, 99%, or 100% identity to at least one of SEQ ID NOS:1-3, or biologically active fragment thereof. The mRNA encoding a G6Pase or a biologically active fragment thereof, therefore, can comprise a nucleotide sequence with at least 70%, 75%, 80%, 85%, 90%, 91%, 92%, 93%, 94%, 95%, 96%, 97%, 98%, 99%, or 100% identity to a nucleotide sequence that encodes at least one of SEQ ID NOS:4-6, or biologically active fragment thereof.

The terms “homology” or “identity” or “similarity” refer to sequence relationships between two nucleic acid molecules and can be determined by comparing a nucleotide position in each sequence when aligned for purposes of comparison. The term “homology” refers to the relatedness of two nucleic acid or protein sequences. The term “identity” refers to the degree to which nucleic acids are the same between two sequences. The term “similarity” refers to the degree to which nucleic acids are the same, but includes neutral degenerate nucleotides that can be substituted within a codon without changing the amino acid identity of the codon, as is well known in the art.

Percent identity can be determined using a sequence alignment tool or program, including but not limited to (1) a BLAST 2.0 Basic BLAST homology search using blastp for amino acid searches and blastn for nucleic acid searches with standard default parameters, wherein the query sequence is filtered for low complexity regions by default; (2) a BLAST 2 alignment (using the parameters described below); (3) PSI BLAST with the standard default parameters (Position Specific Iterated BLAST; (4) and/or Clustal Omega. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches.

One of ordinary skill in the art will recognize that individual substitutions, deletions or additions to a nucleic acid, peptide, polypeptide or protein sequences that alter, add or delete a single amino acid or a small percentage of amino acids in the encoded sequence is a “conservatively modified variant.” Such variants can be useful, for example, to alter the physical properties of the peptide, e.g., to increase stability or efficacy of the peptide. Conservative substitution tables providing functionally similar amino acids are known to those of ordinary skill in the art. Such conservatively modified variants are in addition to and do not exclude polymorphic variants, interspecies homologs and alternate alleles. The following groups provide non limiting examples of amino acids that can be conservatively substituted for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

The term “codon-optimized” refers to genes or coding regions of a nucleic acid molecule to be translated into a polypeptide sequence. Some codons are more commonly used to encode a particular amino acid by particular organisms, and translation efficiency can be improved by changing the mRNA sequence in such a way as the desired codons are effectively used by the desired host translation machinery. This process, where the mRNA sequence is changed to reflect alternate codon usage to improve translation efficiency without affecting the sequence of the translated polypeptide, is referred to as “codon optimization.” One of skill in the art will recognize, that several algorithms are available to codon optimize an mRNA sequence in silico. In particular embodiments, the modified mRNA molecules are codon-optimized.

Codon usage bias refers to differences in the frequency of occurrence of synonymous codons in coding DNA (Hershberg, R. & Petrov, D., Annu. Rev. Genet., 42:287-99, 2008; Eyre-Walker, A. J. Mol. Evol., 33:442-9, 1991). There are 64 different codons (61 codons encoding for amino acids plus 3 stop codons) for only 20 different translated amino acids. The overabundance in the number of codons allows many amino acids to be encoded by more than one codon. Different organisms often show particular preferences for one of the several codons that encode the same amino acid. Codon preferences reflect a balance between mutational biases and natural selection for translational optimization. Optimal codon usage in fast-growing microorganisms, like Escherichia coli or Saccharomyces cerevisiae (baker's yeast), for example, reflects the composition of their respective genomic tRNA pool. Optimal codon usage can achieve faster translation rates and high accuracy.

The modRNA molecules described herein can comprise at least one codon substituted to create the corresponding biased codon specific to the mammal species for delivering such polynucleotide. One exemplary and non-limiting rationale for this substitution is to decrease host immunogenicity and/or to facilitate protein translation in such mammal species. Alternatively, an mRNA can comprise at least one codon substituted to a non-preferred codon in the host mammal species, as such substitutions allow one of skill in the art to attenuate translation speed and efficiency, e.g., to increase differentiation of the expressed protein and/or to add desired properties to the expressed protein or fragment thereof.

### RNA Formation and Modifications

As used herein, the term “nucleic acid” refers to polymeric biomolecules, e.g., genetic material (e.g., oligonucleotides or polynucleotides comprising DNA or RNA), which include any compound and/or substance that comprise a polymer of nucleotides. These polymers are polynucleotides. Nucleic acids described herein include, for example, RNA or stabilized RNA, e.g., modRNA, encoding a protein or enzyme.

The mRNAs described herein can be natural or recombinant, isolated or chemically synthesized. Such mRNAs can be, for example isolated from in vitro cell cultures or from organisms such as plants or animals in vivo. The mRNAs can be, for example, synthesized or produced in silico.

Described herein are compositions and methods for the manufacture and optimization of mRNA molecules, e.g., modRNAs, through modification of the architecture of mRNA molecules. The disclosure provides, for example, methods for increasing production of a G6Pase or a biologically active fragment thereof encoded by the mRNA molecules by altering mRNA sequence and/or structure.

The modRNA can comprise, for example, one or more chemical/structural modifications. Such modification(s) can, for example, reduce the innate immune response of a cell into which the mRNA molecule is introduced or any of plurality of other desired effects including, but not limited to: 1) improving the stability of the mRNA molecule; 2) improving the efficiency of protein production; 3) improving intracellular retention and/or the half-life of the mRNA molecules; and/or 4) improving viability of contacted cells. Exemplary modification methods and compositions can be seen in, for example, PCT publication Nos. WO2014081507, WO2013151664 and WO2007024708, the entire contents of each of which are hereby incorporated by reference.

Provided herein is a modified mRNA molecule containing a translatable region and one, two or more than two different nucleoside modifications. Nucleoside modifications can include, for example, uniform substitution of a ribonucleoside throughout the modRNA, e.g., incorporation of a modified uracil, cytosine, adenine or guanine at every position where uracil, cytosine, adenine or guanine occurs in the mRNA sequence. Alternatively, modifications can occur at specific sequence positions, and thus the modRNA is discreetly modified. In some embodiments, the modRNA exhibits reduced degradation in a cell into which the mRNA is introduced, relative to a corresponding unmodified mRNA. Two or more linked nucleotides, for example, can be inserted, deleted, duplicated, inverted or randomized in the mRNA molecule without significant chemical modification to the mRNA. The chemical modifications can be located on the sugar moiety of an mRNA molecule described herein. The chemical modifications can be located on the phosphate backbone of the mRNA.

The modRNA molecule(s) described herein can be cyclized or concatemerized, to generate a translation competent molecule to assist interactions, for example, between poly-A binding proteins and 5′ end binding proteins. Cyclization or concatemerization can be achieved, for example, by 1) chemical, 2) enzymatic and/or 3) ribozyme catalyzed processes. The newly formed 5′-/3′-linkage can be intramolecular or intermolecular.

modRNA molecules can be, for example, linked using a functionalized linker molecule. A functionalized saccharide molecule, for example, can be chemically modified to contain multiple chemical reactive groups (SH—, NH₂—, N3, etc. . . . ) to react with the cognate moiety on a 3′-functionalized mRNA molecule (e.g., a 3′-maleimide ester, 3′-NHS-ester, alkynyl, etc.). The number of reactive groups on the modified saccharide can be controlled in a stoichiometric fashion to directly control the stoichiometric ratio of conjugated nucleic acid or mRNA.

The mRNA molecule(s) described herein can be conjugated to other polynucleotides, dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases, alkylating agents, phosphate, amino acids, PEG (e.g., PEG-40K), MPEG, [MPEG]₂, radiolabeled markers, enzymes, haptens (e.g., biotin), transport/absorption facilitators (e.g., aspirin, vitamin E, folic acid), synthetic ribonucleases, proteins (e.g., glycoproteins), peptides (e.g., molecules having a specific affinity for a co-ligand), antibodies (e.g., an antibody that binds to a specified cell type such as, for example, a cancer cell, endothelial cell, hepatocyte or bone cell), hormones and hormone receptors, non-peptidic species (such as lipids, lectins, carbohydrates, vitamins, and cofactors), or a drug. Conjugation may result in increased stability and/or half-life and may be particularly useful in targeting the mRNA molecule of the instant disclosure to specific sites in the cell, tissue or organism.

An mRNA molecule described herein can be, for example bi-functional, which means the mRNA molecule has or is capable of two functions, or multi-functional. The multiple functionalities, structural or chemical, can be encoded by the mRNA (e.g., the function may not manifest until the encoded product is translated) or may be a property of the mRNA itself. Similarly, bi-functional mRNA molecules may comprise a function that is covalently or electrostatically associated with the mRNA. Multiple functions may be provided in the context of a complex of a modified RNA and another molecule.

The mRNA molecule can be purified after isolating from a cell, a tissue, or an organism or chemically synthesized. The purification process may include, for example, clean-up, quality assurance, and quality control. Purification may be performed by methods known in the arts such as, for example, chromatographic methods, e.g., using, for example, AGENCOURT® beads (Beckman Coulter Genomics, Danvers, Mass.), poly-T beads, LNA™ oligo-T capture probes (EXIQON® Inc, Vedbaek, Denmark) or HPLC-based purification methods such as, for example, strong anion exchange HPLC, weak anion exchange HPLC, reverse phase HPLC (RP-HPLC), and hydrophobic interaction HPLC (HIC-HPLC). A purified polynucleotide (e.g., mRNA) is present in a form or setting different from that in which it is found in nature or a form or setting different from that in which it existed prior to subjecting it to a treatment or purification method.

A quality assurance and/or quality control check may be conducted using methods such as, but are not limited to, gel electrophoresis, UV absorbance, or analytical HPLC. In another embodiment, the mRNA molecule may be sequenced by methods including, but not limited to, reverse-transcriptase-PCR.

In one embodiment, the mRNA molecule is quantified using methods such as, for example, ultraviolet visible spectroscopy (UV/Vis). The mRNA molecule can be analyzed to determine if the mRNA is of proper size or if degradation has occurred. Degradation of the mRNA can be checked by methods such as, for example, agarose gel electrophoresis, HPLC based purification methods (e.g., strong anion exchange HPLC, weak anion exchange HPLC, reverse phase HPLC (RP-HPLC), and hydrophobic interaction HPLC (HIC-HPLC)), liquid chromatography/mass spectrometry (LCMS), capillary electrophoresis (CE) and capillary gel electrophoresis (CGE).

The described mRNA can comprise at least one structural or chemical modification. The nucleoside that is modified in the mRNA, for example, can be a uridine (U), a cytidine (C), an adenine (A), or guanine (G). The modified nucleoside can be, for example, m⁵C (5-methylcytidine), m⁶A (N6-methyladenosine), s²U (2-thiouridien), ψ (pseudouridine) or Um (2-O-methyluridine). Some exemplary chemical modifications of nucleosides in the mRNA molecule further include, for example, pyridine-4-one ribonucleoside, 5-aza-uridine, 2-thio-5-aza uridine, 2-thiouridine, 4-thio pseudouridine, 2-thio pseudouridine, 5-hydroxyuridine, 3-methyluridine, 5-carboxymethyl uridine, 1-carboxymethyl pseudouridine, 5-propynyl uridine, 1-propynyl pseudouridine, 5-taurinomethyluridine, 1-taurinomethyl pseudouridine, 5-taurinomethyl-2-thio uridine, 1-taurinomethyl-4-thio uridine, 5-methyl uridine, 1-methyl pseudouridine, 4-thio-1-methyl pseudouridine, 2-thio-1-methyl pseudouridine, 1-methyl-1-deaza pseudouridine, 2-thio-1-methyl-1-deaza pseudouridine, dihydrouridine, dihydropseudouridine, 2-thio dihydrouridine, 2-thio dihydropseudouridine, 2-methoxyuridine, 2-methoxy-4-thio uridine, 4-methoxy pseudouridine, 4-methoxy-2-thio pseudouridine, 5-aza cytidine, pseudoisocytidine, 3-methyl cytidine, N4-acetylcytidine, 5-formylcytidine, N4-methylcytidine, 5-hydroxymethylcytidine, 1-methyl pseudoisocytidine, pyrrolo-cytidine, pyrrolo-pseudoisocytidine, 2-thio cytidine, 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebularine, 2-methoxy cytidine, 2-methoxy-5-methyl cytidine, 4-methoxy pseudoisocytidine, 4-methoxy-1-methyl pseudoisocytidine, 2-aminopurine, 2,6-diaminopurine, 7-deaza adenine, 7-deaza-8-aza adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N⁶-methyladenosine, N⁶-isopentenyladenosine, N⁶-(cis-hydroxyisopentenyl) adenosine, 2-methylthio-N⁶-(cis-hydroxyisopentenyl) adenosine, N⁶-glycinylcarbamoyladenosine, N⁶-threonylcarbamoyladenosine, 2-methylthio-N⁶-threonyl carbamoyladenosine, N⁶,N⁶-dimethyladenosine, 7-methyladenine, 2-methylthio adenine, 2-methoxy adenine, inosine, 1-methyl inosine, wyosine, wybutosine, 7-deaza guanosine, 7-deaza-8-aza guanosine, 6-thio guanosine, 6-thio-7-deaza guanosine, 6-thio-7-deaza-8-aza guanosine, 7-methyl guanosine, 6-thio-7-methyl guanosine, 7-methylinosine, 6-methoxy guanosine, 1-methylguanosine, N²-methylguanosine, N²,N²-dimethylguanosine, 8-oxo guanosine, 7-methyl-8-oxo guanosine, 1-methyl-6-thio guanosine, N²-methyl-6-thio guanosine, and N²,N²-dimethyl-6-thio guanosine. In another embodiment, the modifications are independently selected from the group consisting of 5-methylcytosine, pseudouridine and 1-methylpseudouridine.

In some embodiments, the modified nucleobase in the mRNA molecule is a modified uracil including, for example, pseudouridine (ψ), pyridine-4-one ribonucleoside, 5-aza uridine, 6-aza uridine, 2-thio-5-aza uridine, 2-thio uridine (s2U), 4-thio uridine (s4U), 4-thio pseudouridine, 2-thio pseudouridine, 5-hydroxy uridine (ho⁵U), 5-aminoallyl uridine, 5-halo uridine (e.g., 5-iodom uridine or 5-bromo uridine), 3-methyl uridine (m³U), 5-methoxy uridine (mo⁵U), uridine 5-oxyacetic acid (cmo⁵U), uridine 5-oxyacetic acid methyl ester (mcmo⁵U), 5-carboxymethyl uridine (cm⁵U), 1-carboxymethyl pseudouridine, 5-carboxyhydroxymethyl uridine (chm⁵U), 5-carboxyhydroxymethyl uridine methyl ester (mchm⁵U), 5-methoxycarbonylmethyl uridine (mcm⁵U), 5-methoxycarbonylmethyl-2-thio uridine (mcm⁵s2U), 5-aminomethyl-2-thio uridine (nm⁵s2U), 5-methylaminomethyl uridine (mnm⁵U), 5-methylaminomethyl-2-thio uridine (mnm⁵s2U), 5-methylaminomethyl-2-seleno uridine (mnm⁵se²U), 5-carbamoylmethyl uridine (ncm⁵U), 5-carboxymethylaminomethyl uridine (cmnm⁵U), 5-carboxymethylaminomethyl-2-thio uridine (cmnm⁵s2U), 5-propynyl uridine, 1-propynyl pseudouridine, 5-taurinomethyl uridine (τcm⁵U), 1-taurinomethyl pseudouridine, 5-taurinomethyl-2-thio uridine (τm⁵s2U), 1-taurinomethyl-4-thio pseudouridine, 5-methyl uridine (m⁵U, e.g., having the nucleobase deoxythymine), 1-methyl pseudouridine (m¹′ψ), 5-methyl-2-thio uridine (m⁵s2U), 1-methyl-4-thio pseudouridine (m's⁴ψ), 4-thio-1-methyl pseudouridine, 3-methyl pseudouridine (m³ψ), 2-thio-1-methyl pseudouridine, 1-methyl-1-deaza pseudouridine, 2-thio-1-methyl-1-deaza pseudouridine, dihydrouridine (D), dihydropseudouridine, 5,6-dihydrouridine, 5-methyl dihydrouridine (m⁵D), 2-thio dihydrouridine, 2-thio dihydropseudouridine, 2-methoxy uridine, 2-methoxy-4-thio uridine, 4-methoxy pseudouridine, 4-methoxy-2-thio pseudouridine, N¹-methyl pseudouridine, 3-(3-amino-3-carboxypropyl) uridine (acp³U), 1-methyl-3-(3-amino-3-carboxypropyl) pseudouridine (acp³ψ), 5-(isopentenylaminomethyl) uridine (inm⁵U), 5-(isopentenylaminomethyl)-2-thio uridine (inm⁵s2U), .alpha-thio uridine, 2′-O-methyl uridine (Um), 5,2′-O-dimethyl uridine (m⁵Um), 2′-O-methyl pseudouridine (ψm), 2-thio-2′-O-methyl uridine (s2Um), 5-methoxycarbonylmethyl-2′-O-methyl uridine (mcm⁵Um), 5-carbamoylmethyl-2′-O-methyl uridine (ncm⁵Um), 5-carboxymethylaminomethyl-2′-O-methyl uridine (cmnm⁵Um), 3,2′-O-dimethyl uridine (m³Um), 5-(isopentenylaminomethyl)-2′-O-methyl uridine (inm⁵Um), 1-thio uridine, deoxythymidine, 2′-F-ara uridine, 2′-F uridine, 2′-OH-ara uridine, 5-(2-carbomethoxyvinyl) uridine, and 5-[3-(1-E-propenylamino) uridine.

In some embodiments, the modified nucleobase is a modified cytosine including, for example, 5-aza cytidine, 6-aza cytidine, pseudoisocytidine, 3-methyl cytidine (m³C), N⁴-acetyl cytidine (act), 5-formyl cytidine (f⁵C), N⁴-methyl cytidine (m⁴C), 5-methyl cytidine (m⁵C), 5-halo cytidine (e.g., 5-iodo cytidine), 5-hydroxymethyl cytidine (hm⁵C), 1-methyl pseudoisocytidine, pyrrolo-cytidine, pyrrolo-pseudoisocytidine, 2-thio cytidine (s2C), 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebularine, 2-methoxy cytidine, 2-methoxy-5-methyl cytidine, 4-methoxy pseudoisocytidine, 4-methoxy-1-methyl pseudoisocytidine, lysidine (k²C), alpha-thio cytidine, 2′-O-methyl cytidine (Cm), 5,2′-O-dimethyl cytidine (m⁵Cm), N⁴-acetyl-2′-O-methyl cytidine (ac⁴Cm), N⁴,2′-O-dimethyl cytidine (m⁴Cm), 5-formyl-2′-O-methyl cytidine (f⁵Cm), N⁴,N⁴,2′-O-trimethyl cytidine (m⁴²Cm), 1-thio cytidine, 2′-F-ara cytidine, 2′-F cytidine, and 2′-OH-ara cytidine.

In some embodiments, the modified nucleobase is a modified adenine including, for example, 2-amino purine, 2,6-diamino purine, 2-amino-6-halo purine (e.g., 2-amino-6-chloro purine), 6-halo purine (e.g., 6-chloro purine), 2-amino-6-methyl purine, 8-azido adenosine, 7-deaza adenine, 7-deaza-8-aza adenine, 7-deaza-2-amino purine, 7-deaza-8-aza-2-amino purine, 7-deaza-2,6-diamino purine, 7-deaza-8-aza-2,6-diamino purine, 1-methyl adenosine (m¹A), 2-methyl adenine (m²A), N⁶-methyl adenosine (m⁶A), 2-methylthio-N⁶-methyl adenosine (ms²m⁶A), N⁶-isopentenyl adenosine (i⁶A), 2-methylthio-N⁶-isopentenyl adenosine (ms²i⁶A), N⁶-(cis-hydroxyisopentenyl) adenosine (io⁶A), 2-methylthio-N⁶-(cis-hydroxyisopentenyl) adenosine (ms²io⁶A), N⁶-glycinylcarbamoyl adenosine (g⁶A), N⁶-threonylcarbamoyl adenosine (t⁶A), N⁶-methyl-N⁶-threonylcarbamoyl adenosine (m⁶t⁶A), 2-methylthio-N⁶-threonylcarbamoyl adenosine (ms²g⁶A), N⁶,N⁶-dimethyl adenosine (m⁶₂A), N⁶-hydroxynorvalylcarbamoyl adenosine (hn⁶A), 2-methylthio-N⁶-hydroxynorvalylcarbamoyl adenosine (ms²hn⁶A), N⁶-acetyl adenosine (ac⁶A), 7-methyl adenine, 2-methylthio adenine, 2-methoxy adenine, alpha-thio adenosine, 2′-O-methyl adenosine (Am), N⁶,2′-O-dimethyl adenosine (m⁶Am), N⁶,N⁶,2′-O-trimethyl adenosine (m⁶²Am), 1,2′-O-dimethyl adenosine (m¹Am), 2′-O-ribosyl adenosine (phosphate) (Ar(p)), 2-amino-N⁶-methyl purine, 1-thio adenosine, 8-azido adenosine, 2′-F-ara adenosine, 2′-F adenosine, 2′-OH-ara adenosine, and N⁶-(19-amino-pentaoxanonadecyl) adenosine.

In some embodiments, the modified nucleobase is a modified guanine including, for example, inosine (I), 1-methyl inosine (m¹I), wyosine (imG), methylwyosine (mimG), 4-demethyl wyosine (imG-14), isowyosine (imG2), wybutosine (yW), peroxywybutosine (o₂yW), hydroxywybutosine (OHyW), undermodified hydroxywybutosine (OHyWy), 7-deaza guanosine, queuosine (Q), epoxyqueuosine (oQ), galactosyl queuosine (galQ), mannosyl queuosine (manQ), 7-cyano-7-deaza guanosine (preQ₀), 7-aminomethyl-7-deaza guanosine (preQ₁), archaeosine (G+), 7-deaza-8-aza guanosine, 6-thio guanosine, 6-thio-7-deaza guanosine, 6-thio-7-deaza-8-aza guanosine, 7-methyl guanosine (m⁷G), 6-thio-7-methyl guanosine, 7-methyl inosine, 6-methoxy guanosine, 1-methyl guanosine (m¹G), N²-methyl-guanosine (m²G), N²,N²-dimethyl guanosine (m²₂G), N^{2,7}-dimethyl guanosine (m^{2,7}G), N², N^{2,7}-dimethyl guanosine (m^{2,2,7}G), 8-oxo guanosine, 7-methyl-8-oxo guanosine, 1-methio guanosine, N²-methyl-6-thio guanosine, N²,N²-dimethyl-6-thio guanosine, alpha-thio guanosine, 2′-O-methyl guanosine (Gm), N²-methyl-2′-O-methyl guanosine (m²Gm), N²,N²-dimethyl-2′-O-methyl guanosine (m²₂Gm), 1-methyl-2′-O-methyl guanosine (m¹Gm), N^{2,7}-dimethyl-2′-O-methyl guanosine (m^{2,7}Gm), 2′-O-methyl inosine (Im), 1,2′-O-dimethyl inosine (m¹Im), 2′-O-ribosyl guanosine (phosphate) (Gr(p)), 1-thio guanosine, O⁶-methyl guanosine, 2′-F-ara guanosine, and 2′-F guanosine.

The nucleobase of the nucleotide can be independently selected from a purine, a pyrimidine, a purine or pyrimidine analog. For example, the nucleobase can each be independently selected from adenine, cytosine, guanine, uracil or hypoxanthine. The nucleobase can also include, for example, naturally occurring and synthetic derivatives of a base, including, but not limited to, pyrazolo[3,4-d]pyrimidines, 5-methylcytosine (5-me-C), 5-hydroxymethyl cytosine, xanthine, hypoxanthine, 2-amino adenine, 6-methyl and other alkyl derivatives of adenine and guanine, 2-propyl and other alkyl derivatives of adenine and guanine, 2-thio uracil, 2-thio thymine and 2-thio cytosine, 5-propynyl uracil and cytosine, 6-azo uracil, cytosine and thymine, pseudouracil, 4-thio uracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methyl guanine and 7-methyl adenine, 8-aza guanine and 8-aza adenine, deaza guanine, 7-deaza guanine, 3-deaza guanine, deaza adenine, 7-deaza adenine, 3-deaza adenine, pyrazolo[3,4-d]pyrimidine, imidazo[1,5-a]1,3,5 triazinones, 9-deaza purines, imidazo[4,5-d]pyrazines, thiazolo[4,5-d]pyrimidines, pyrazine-2-ones, 1,2,4-triazine, pyridazine; and 1,3,5-triazine. When the nucleotides are depicted using the shorthand A, G, C, T or U, each letter refers to the representative base and/or derivatives thereof, e.g., A includes adenine or adenine analogs, e.g., 7-deaza adenine).

Other modifications include, for example, those in U.S. Pat. No. 8,835,108; U.S. Patent Application Publication No. 20130156849; Tavernier, G. et al., J. Control. Release, 150:238-47, 2011; Anderson, B. et al., Nucleic Acids Res., 39:9329-38, 2011; Kormann, M. et al., Nat. Biotechnol., 29:154-7, 2011; Karikó, K. et al., Mol. Ther., 16:1833-40, 2008; Karikó, K. et al., Immunity, 23:165-75, 2005; and Warren, L. et al., Cell Stem Cell, 7:618-30, 2010; the entire contents of each of which is incorporated herein by reference.

### Compositions

The mRNA described herein can be delivered into a host, such as a mammal (e.g., a human), to express a protein of interest (e.g., a G6Pase or biologically active fragment thereof). The mRNA can comprise an exon of the protein of interest for in vivo expression. Optionally, the mRNA can have at least one of the introns of the protein of interest or another protein to facilitate gene expression. For the encoded G6Pase or biologically active fragment(s) thereof, different subunit polypeptides or domains of the same or different subunit polypeptides can be expressed from a single mRNA molecule or from two different mRNA molecules (e.g., each chain expressing a different subunit). In latter situation these two mRNA molecules can be co-delivered into the host for in vivo expression. Optionally, the one or two mRNA molecule can be delivered in conjunction with a polypeptide or protein, or an mRNA encoding such polypeptide or protein, which is capable of facilitating protein expression of the G6Pase or biologically active fragments thereof (e.g., co-expression of one or more biologically active fragments).

### Delivery

When formulated in a nanoparticle for delivery, modified mRNA show increased nuclease tolerance and is more effectively taken up by tumor cells after systemic administration (Wang, Y. et al., Mol. Ther., 21:358-67, 2013; the content of which is incorporated by reference herein in its entirety). mRNA can be delivered, for example, by multiple methods to the host organism (PCT publication Nos: WO2013185069, WO2012075040 and WO2011068810, the entire contents of each of which are herein incorporated by reference).

Lipid carrier vehicles can be used to facilitate the delivery of nucleic acids to target cells. Lipid carrier vehicles (e.g., liposomes and lipid-derived nanoparticles (LNPs), such as, for example, the MC3 LNP (Arbutus Biopharma)) are generally useful in a variety of applications in research, industry, and medicine, particularly for their use as transfer vehicles of diagnostic or therapeutic compounds in vivo (Lasic, D., Trends Biotechnol., 16:3-7-21, 1998; Drummond, D. et al., Pharmacol. Rev., 51:691-743, 1999) and are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains.

The liposomal transfer vehicles are prepared to contain the desired nucleic acids for the protein of interest. The process of incorporation of a desired entity (e.g., a nucleic acid such as, for example, an mRNA) into a liposome is referred to as “loading” (Lasic, D. et al., FEBS Lett., 312:255-8, 1992). The liposome-incorporated nucleic acids can be completely or be partially located in the interior space of the liposome, within the bilayer membrane of the liposome, or associated with the exterior surface of the liposome membrane. The incorporation of a nucleic acid into liposomes is referred to herein as “encapsulation,” wherein the nucleic acid is entirely contained within the interior space of the liposome. The purpose of incorporating an mRNA into a transfer vehicle, such as a liposome, is often to protect the nucleic acid from an environment that may contain enzymes or chemicals that degrade nucleic acids and/or systems or receptors that cause the rapid excretion of the nucleic acids. Accordingly, the selected transfer vehicle is capable of enhancing the stability of the mRNA contained therein. The liposome allows the encapsulated mRNA to reach a desired target cell.

As used herein, the term “target cell” refers to a cell or tissue to which a composition described herein is to be directed or targeted. In some embodiments, the target cells are deficient in a protein or enzyme of interest. For example, where it is desired to deliver a nucleic acid to a hepatocyte, the hepatocyte represents the target cell. In some embodiments, the nucleic acids and compositions specifically transfect the target cells (i.e., they do not transfect non-target cells). The compositions and methods can be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

The compositions described herein can be administered and dosed in accordance with current medical practice, taking into account, for example, the clinical condition of the subject, the site and method of administration, the scheduling of administration, the subject's age, sex, body weight and other factors relevant to clinicians of ordinary skill in the art. The “effective amount” for the purposes herein may be determined by such relevant considerations as are known to those of ordinary skill in experimental clinical research, pharmacological, clinical and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient expression of the antibody or fragment in the target cell.

The route of delivery used in the methods of the disclosure allows for noninvasive, self-administration of the therapeutic compositions of mRNA described herein. The methods involve intratracheal or pulmonary administration by aerosolization, nebulization, or instillation of compositions comprising the mRNA in a suitable transfection or lipid carrier vehicles as described herein.

Following administration of the composition to the subject, the protein of interest, e.g., G6Pase or biologically active fragment(s) thereof encoded by the mRNA, is detectable in the target tissues for at least about one to about seven days or longer following administration of the composition to the subject. The amount of expressed protein or protein fragment necessary to achieve a therapeutic effect varies depending on the condition being treated and the condition of the patient. The expressed G6Pase or fragment(s), for example, is detectable in the target tissues at a concentration of at least 0.025-1.5 μg/mL (e.g., at least 0.050 μg/mL, at least 0.075 μg/mL, at least 0.1 μg/mL, at least 0.2 μg/mL, at least 0.3 μg/mL, at least 0.4 μg/mL, at least 0.5 μg/mL, at least 0.6 μg/mL, at least 0.7 μg/mL, at least 0.8 μg/mL, at least 0.9 μg/mL, at least 1.0 μg/mL, at least 1.1 μg/mL, at least 1.2 μg/mL, at least 1.3 μg/mL, at least 1.4 μg/mL, or at least 1.5 μg/mL), or at a higher concentration, for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40 or 45 days or longer following administration of the composition to the subject.

### Pharmaceutical Compositions and Formulations

The mRNA compositions described herein can be formulated as a pharmaceutical solution, e.g., for administration to a subject for the treatment or prevention of a disease or disorder associated with G6Pase deficiency, e.g., GSDIa. The pharmaceutical compositions can include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (Berge. S. et al., J. Pharm. Sci., 66:1-19, 1977).

The compositions can be formulated according to methods in the art (Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X)). A composition can be formulated, for example, as a buffered solution at a suitable concentration and suitable for storage at 2-8C (e.g., 4C). In some embodiments, a composition can be formulated for storage at a temperature below 0C (e.g., −20C or −80C). In some embodiments, the composition can be formulated for storage for up to two years (e.g., one month, two months, three months, four months, five months, six months, seven months, eight months, nine months, 10 months, 11 months, 1 year, 1½ years or 2 years). Thus, in some embodiments, the compositions described herein are stable in storage for at least one year at 2-8C (e.g., 4C).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. For example, compositions containing an mRNA molecule intended for systemic or local delivery can be in the form of injectable or infusible solutions. Accordingly, the compositions can be formulated for administration by a parenteral mode (e.g., intravenous, subcutaneous, intraperitoneal or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

The compositions can be formulated as a solution, microemulsion, dispersion, liposome or other ordered structure suitable for stable storage at high concentration. Sterile injectable solutions can be prepared by incorporating a composition described herein in the required amount in an appropriate solvent with one or a combination of ingredients enumerated above, as required or otherwise desirable, followed by filter sterilization. Dispersions are generally prepared by incorporating a composition into a sterile vehicle that contains a basic dispersion medium and other ingredients from those enumerated above. In the case of sterile powders for the preparation of sterile injectable solutions, methods for preparation include vacuum drying and freeze-drying that yield a powder of a composition plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts and gelatin.

The mRNA compositions described herein can also be formulated in liposome compositions prepared by methods known in the art (e.g., Eppstein, D. et al., Proc. Natl. Acad. Sci. USA, 82:3688-92, 1985; Hwang, K. et al., Proc. Natl. Acad. Sci. USA, 77:4030-4, 1980; and U.S. Pat. Nos. 4,485,045; 4,544,545 and 5,013,556; the entire contents of each of which is incorporated by reference herein).

Compositions can be formulated with a carrier, for example, which protects the formulated mRNA against rapid release, such as a controlled release formulation, including implants and microencapsulated delivery systems. Biodegradable, biocompatible polymers, for example, can be used (e.g., ethylene vinyl acetate, polyanhydrides, polyglycolic acid, collagen, polyorthoesters and polylactic acid). Many methods for the preparation of such formulations are known in the art (e.g., J.R. Robinson (1978) “Sustained and Controlled Release Drug Delivery Systems,” Marcel Dekker, Inc., New York).

Compositions can be formulated for delivery to the eye. As used herein, the term “eye” refers to any and all anatomical tissues and structures associated with an eye.

In some embodiments, compositions can be administered locally, for example, by way of topical application or intravitreal injection. For example, in some embodiments, the compositions can be formulated for administration by way of an eye drop.

The therapeutic preparation for treating the eye can contain one or more active agents in a concentration from about 0.01 to about 1% by weight, preferably from about 0.05 to about 0.5% in a pharmaceutically acceptable solution, suspension or ointment. The preparation can be, for example, in the form of a sterile aqueous solution containing, e.g., additional ingredients such as, but are not limited to, preservatives, buffers, tonicity agents, antioxidants and stabilizers, nonionic wetting or clarifying agents and viscosity-increasing agents.

Suitable preservatives for use in such a solution include, for example, benzalkonium chloride, benzethonium chloride, chlorobutanol, thimerosal and the like. Suitable buffers include, e.g., boric acid, sodium and potassium bicarbonate, sodium and potassium borates, sodium and potassium carbonate, sodium acetate, and sodium biphosphate, in amounts sufficient to maintain the pH at between about pH 6 and about pH 8, and preferably, between pH 7 and pH 7.5. Suitable tonicity agents include, for example, dextran 40, dextran 70, dextrose, glycerin, potassium chloride, propylene glycol and sodium chloride.

Suitable antioxidants and stabilizers include, for example, sodium bisulfite, sodium metabisulfite, sodium thiosulfite and thiourea. Suitable wetting and clarifying agents include, for example, polysorbate 80, polysorbate 20, poloxamer 282 and tyloxapol. Suitable viscosity-increasing agents include, for example, dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone and carboxymethylcellulose.

As described above, relatively high concentration (mRNA) compositions can be made. For example, the compositions can be formulated at an mRNA concentration between about 10 mg/mL to about 100 mg/mL (e.g., between about 9 mg/mL and about 90 mg/mL; between about 9 mg/mL and about 50 mg/mL; between about 10 mg/mL and about 50 mg/mL; between about 15 mg/mL and about 50 mg/mL; between about 15 mg/mL and about 110 mg/mL; between about 15 mg/mL and about 100 mg/mL; between about 20 mg/mL and about 100 mg/mL; between about 20 mg/mL and about 80 mg/mL; between about 25 mg/mL and about 100 mg/mL; between about 25 mg/mL and about 85 mg/mL; between about 20 mg/mL and about 50 mg/mL; between about 25 mg/mL and about 50 mg/mL; between about 30 mg/mL and about 100 mg/mL; between about 30 mg/mL and about 50 mg/mL; between about 40 mg/mL and about 100 mg/mL; or between about 50 mg/mL and about 100 mg/mL). In some embodiments, compositions can be formulated at a concentration of greater than 5 mg/mL and less than 50 mg/mL. Methods for formulating a protein in an aqueous solution are known in the art, e.g., U.S. Pat. No. 7,390,786; McNally and Hastedt (2007), “Protein Formulation and Delivery,” Second Edition, Drugs and the Pharmaceutical Sciences, Volume 175, CRC Press; and Banga (2005), “Therapeutic peptides and proteins: formulation, processing, and delivery systems, Second Edition” CRC Press.

In some embodiments, the aqueous solution has a neutral pH, e.g., a pH between, e.g., 6.5 and 8 (e.g., between and inclusive of 7 and 8). In some embodiments, the aqueous solution has a pH of about 6.6, 6.7, 6.8, 6.9, 7, 7.1, 7.2, 7.3, 7.4, 7.5, 7.6, 7.7, 7.8, 7.9 or 8.0. In some embodiments, the aqueous solution has a pH of greater than (or equal to) 6 (e.g., greater than or equal to 6.1, 6.2, 6.3, 6.4, 6.5, 6.6, 6.7, 6.8, 6.9, 7, 7.1, 7.2, 7.3, 7.4, 7.5, 7.6, 7.7, 7.8 or 7.9), but less than pH 8.

In some embodiments, compositions can be formulated with one or more additional therapeutic agents, e.g., additional therapies for treating or preventing a disease or disorder described herein, e.g., G6Pase-deficiency-associated disease or disorder in a subject. When compositions are to be used in combination with a second active agent, the compositions can be co-formulated with the second agent or the compositions can be formulated separately from the second agent formulation. The respective pharmaceutical compositions can be mixed, for example, just prior to administration, and administered together or can be administered separately, e.g., at the same or different times.

### EXAMPLE

The Examples that follow are illustrative of specific embodiments of the invention, and various uses thereof. They are set forth for explanatory purposes only, and should not be construed as limiting the scope of the invention in any way.

### Example 1. Expression and Activity of G6PC mRNA Constructs

A mouse model for GSD1a was used to evaluate therapeutic agents to correct G6Pase activity. For the mouse model, animal survival is normal due to G6Pase activity in the kidney. Liver glycogen, G6P and triglycerides are relevant biomarkers for monitoring G6Pase activity in the liver. Fasting blood glucose at earlier time points was used as a key biomarker, and liver weight has proven to be a key indicator of disease burden.

| Construct | Substitutions |
|---|---|
| GFP | control- n.a. |
| hG6PC (WT) | none |
| hG6PC Variant 1 | S297G, S298C, V308L, V321I, V340L |
| hG6PC Variant 2 | Q139R, I142K, Q247R, S298C, V308L |

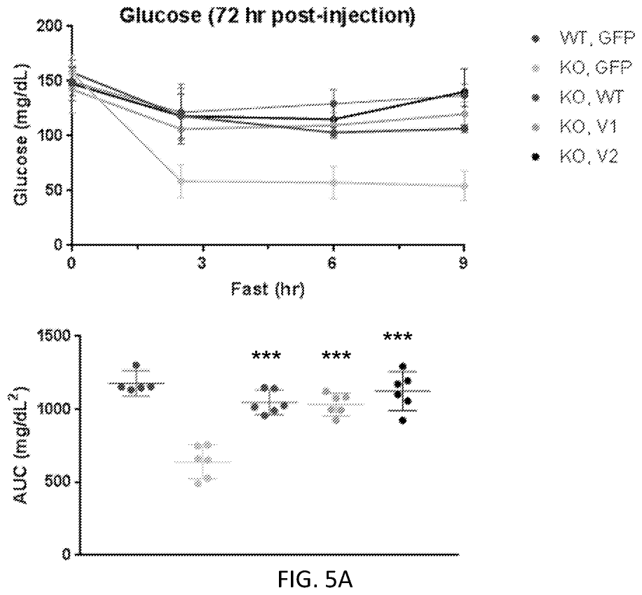

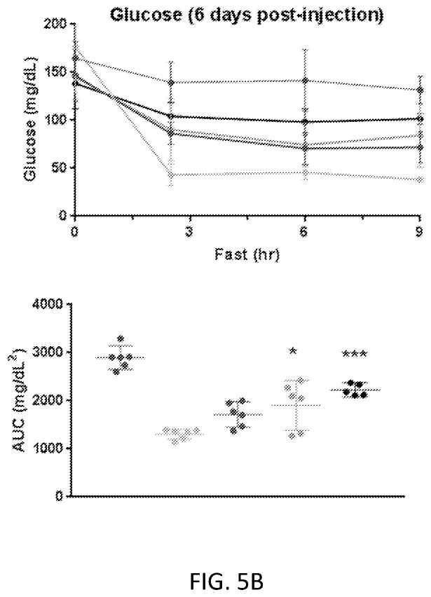

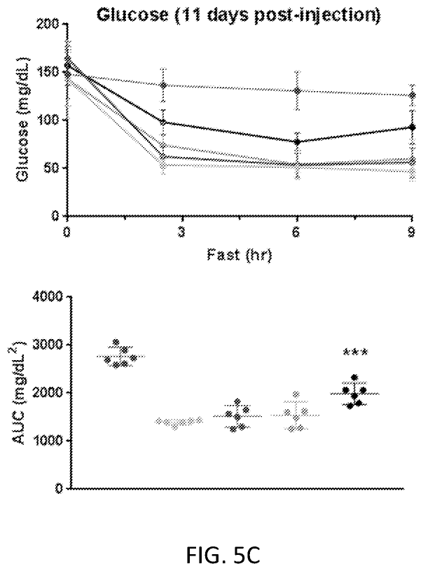

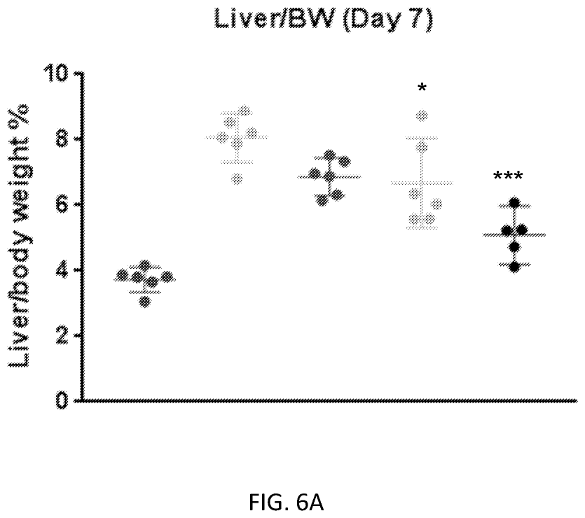

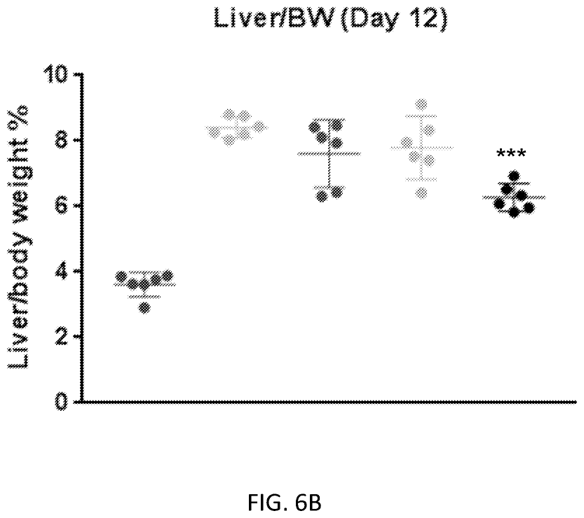

A G6PC sequence, Variant 2, was able to improve fasting glucose levels at >11 days post-dose and significantly reduce liver weight at 12 days in a GSD1a mouse model (FIGS. 5C and 6B).

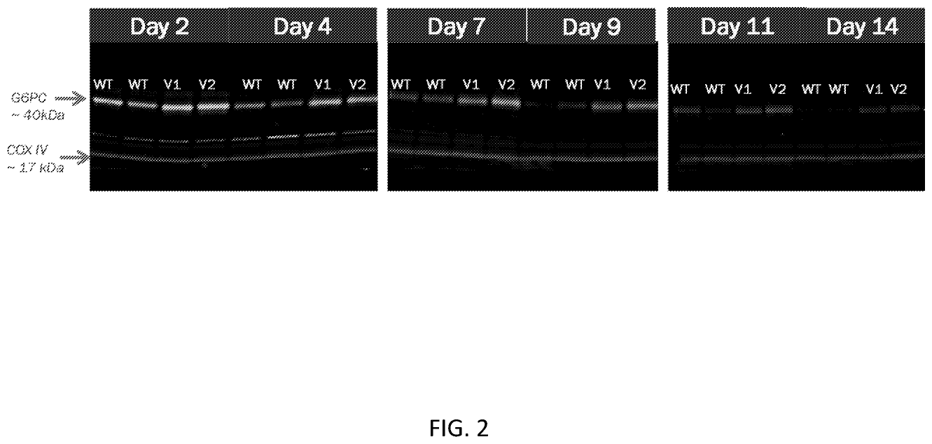

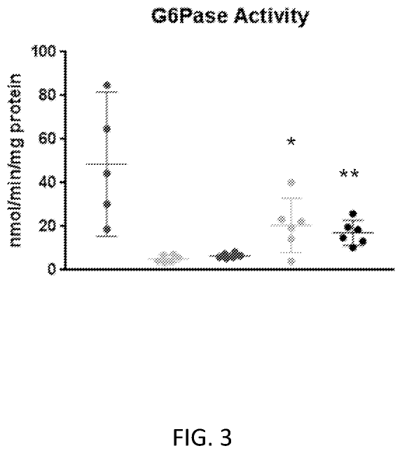

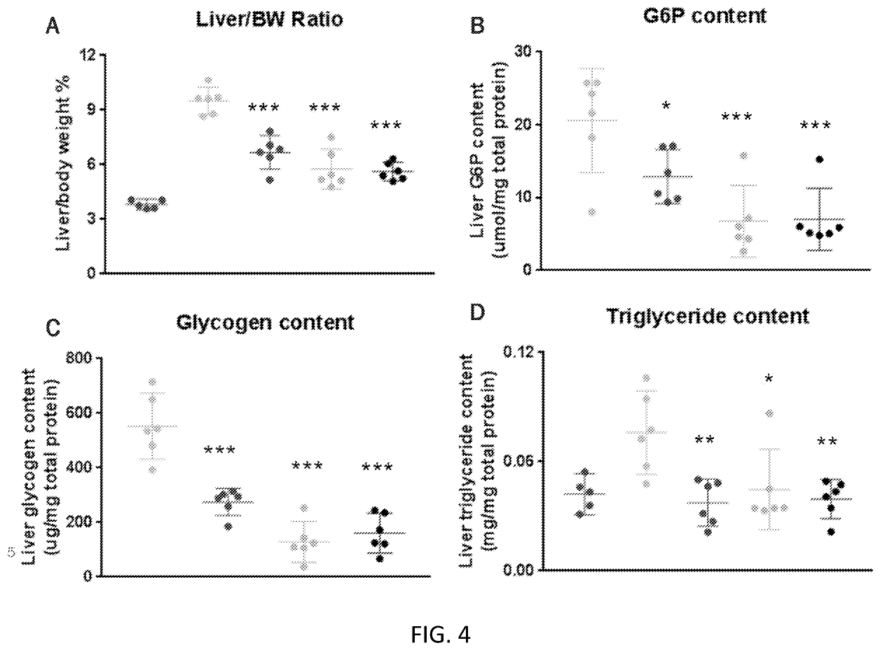

G6Pase activity was measured, for example, in HEK293 cells cultured on a 6-well plate (4×10⁵ cells per well). The cells were transfected with 1 μg of G6PC mRNA (wild-type or variant) or control using lipofectamine 2000. Cells were lysed, and protein expression was determined by capillary electrophoresis (CE) or SDS-PAGE (FIG. 2), and G6Pase activity was determined using an in vitro assay (FIG. 3).

### OTHER EMBODIMENTS

It is understood that while the invention has been described in conjunction with the detailed description thereof, the foregoing description is intended to illustrate and not limit the scope of the invention, which is defined by the scope of the appended claims. The materials, methods, and examples are illustrative only and not intended to be limiting. All publications, patent applications, patents, sequences, database entries and other references cited and described herein are incorporated by reference in their entireties. Other aspects, advantages and modifications are within the scope of the following claims.

What is claimed is:

1. A method of treating a disease or disorder associated with a glucose-6-phosphatase deficiency in a subject comprising administering to the subject a therapeutically effective amount of a composition comprising a modified mRNA molecule encoding a glucose-6-phosphatase polypeptide or active fragment thereof.

2. The method of claim 1, wherein the glucose-6-phosphatase polypeptide is encoded by G6PC.

3. The method of claim 2, wherein the glucose-6-phosphatase polypeptide comprises an amino acid sequence that is at least about 80% identical to a sequence selected from the group consisting of SEQ ID NOS:4-6.

4. The method of claim 2, wherein the glucose-6-phosphatase polypeptide comprises an amino acid sequence that is at least 85% identical to a sequence selected from the group consisting of SEQ ID NOS:4-6.

5. The method of claim 2, wherein the glucose-6-phosphatase polypeptide comprises an amino acid sequence that is at least 90% identical to a sequence selected from the group consisting of SEQ ID NOS:4-6.

6. The method of claim 2, wherein the glucose-6-phosphatase polypeptide comprises an amino acid sequence that is at least 95% identical to a sequence selected from the group consisting of SEQ ID NOS:1-3.

7. The method of claim 2, wherein the glucose-6-phosphatase polypeptide comprises an amino acid sequence selected from the group consisting of SEQ ID NOS:1-3.

8. The method of claim 1, wherein the modified mRNA molecule comprises a sequence complementary to a nucleotide sequence that is at least about 80% identical to a sequence selected from the group consisting of SEQ ID NOS:4-6.

9. The method of claim 1, wherein the modified mRNA molecule comprises a sequence complementary to a nucleotide sequence that is at least 85% identical to a sequence selected from the group consisting of SEQ ID NOS:4-6.

10. The method of claim 1, wherein the modified mRNA molecule comprises a sequence complementary to a nucleotide sequence that is at least 90% identical to a sequence selected from the group consisting of SEQ ID NOS:4-6.

11. The method of claim 1, wherein the modified mRNA molecule comprises a sequence complementary to a nucleotide sequence that is at least 95% identical to a sequence selected from the group consisting of SEQ ID NOS:4-6.

12. The method of claim 1, wherein the modified mRNA molecule comprises a sequence complementary to the nucleotide sequence selected from the group consisting of SEQ ID NOS:4-6.

13. The method of claim 1, wherein the glucose-6-phosphatase deficiency is glycogen storage disease type 1a (von Gierke's disease).

14. The method of claim 1, wherein the modified mRNA molecule comprises at least one modified nucleoside selected from the group consisting of: pseudouridine, 1-methyl pseudouridine, N1-methyl pseudouridine, 5-methylcytidine, 5-methyluridine, 2′-O-methyluridine, 2-thiouridine and N⁶-methyladenosine.

15. The method of claim 1, wherein the modified mRNA molecule comprises a poly(A) tail, a Kozak sequence, a 3′ untranslated region, a 5′ untranslated region or any combination thereof.

16. A pharmaceutical composition comprising a therapeutically effective amount of a modified mRNA molecule encoding a glucose-6-phosphatase polypeptide, active variant of a glucose-6-phosphatase polypeptide, or active fragment thereof formulated in a lipid nanoparticle carrier.

17. A method of reducing glycogen levels in a subject comprising administering a therapeutically effective amount of a modified mRNA capable of expressing a glucose-6-phosphatase polypeptide or biologically active fragment thereof.

18. The method of claim 17, wherein the glucose-6-phosphatase polypeptide is encoded by G6PC.

19. The method of claim 18, wherein the glucose-6-phosphatase polypeptide comprises an amino acid sequence that is at least about 80% identical to a sequence selected from the group consisting of SEQ ID NOS:1-3.

20. The method of claim 18, wherein the glucose-6-phosphatase polypeptide comprises an amino acid sequence that is at least 85% identical to a sequence selected from the group consisting of SEQ ID NOS:1-3.

21. The method of claim 18, wherein the glucose-6-phosphatase polypeptide comprises an amino acid sequence that is at least 90% identical to a sequence selected from the group consisting of SEQ ID NOS:1-3.

22. The method of claim 18, wherein the glucose-6-phosphatase polypeptide comprises an amino acid sequence that is at least 95% identical to a sequence selected from the group consisting of SEQ ID NOS:1-3.
